(19) United States
(12) Patent Application Publication
(10) Pub. No.: US 2026/0069592 A1
(43) Pub. Date: Mar. 12, 2026

(54) USE OF A QUINAZOLINE COMPOUND IN OVERCOMING OSIMERTINIB RESISTANCE

(71) Applicant: WEISHANG (SHANGHAI) BIO-PHARMACEUTICAL CO., LTD., Shanghai (CN)

(72) Inventor: Wei ZHONG, Corona, CA (US)

(21) Appl. No.: 19/107,512

(22) PCT Filed: Feb. 5, 2024
(86) PCT No.: PCT/CN2024/076045

§ 371 Date: Feb. 27, 2025

(30) Foreign Application Priority Data
Jan. 16, 2023 (CN) 202310079445.2

(51) Int. Cl. A61K 31/517; A61P 35/00

(52) U.S. Cl. CPC A61K 31/517; A61P 35/00

(57) ABSTRACT

The present invention provides the use of a quinazoline compound in overcoming osimertinib resistance. The quinazoline compound includes the quinazoline derivative of formula I, at least one of its salts, prodrugs, and prodrug salts, and at least one solvate, hydrate, and polymorph of the said salts.

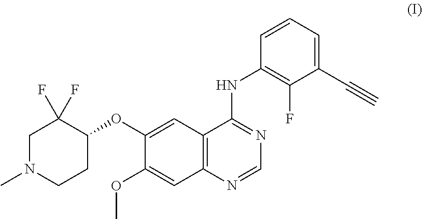 [SMILES: C#Cc1cccc(Nc2ncnc3cc(OC)c(O[C@@H]4CCN(C)CC4(F)F)cc23)c1F]

Compared with the prior art, the present invention has the following advantageous effects: 1. The quinazoline derivative (I) and pharmaceutically acceptable salts thereof described in this invention can inhibit tumor growth by overcoming osimertinib resistance especially caused by the EGFR C797S mutation, L792H mutation, Del19/C797S dual mutations, or L858R/C797S dual mutations. 2. The quinazoline derivative (I) and pharmaceutically acceptable salts thereof described in the present invention can effectively treat non-small cell lung cancer (NSCLC) that is unresponsive to osimertinib treatment, including NSCLC with central nervous system (CNS) metastases and cancers driven by the aforementioned resistance mutations.

### TECHNICAL FIELD

The present invention relates to the use of a quinazoline compound in overcoming osimertinib resistance, belonging to the field of biopharmaceutical technology.

### BACKGROUND ART

The quinazoline derivative with the molecular formula C₂₃H₂₁F₃N₄O₂, chemical name (R)-6-[(3,3-difluoro-1-methylpiperidin-4-yl)oxy]-N-(3-ethynyl-2-fluorophenyl)-7-methoxyquinazolin-4-amine (I), is a targeted anticancer drug capable of crossing the blood-brain barrier. It is a highly selective epidermal growth factor receptor (EGFR) tyrosine kinase inhibitor and can be used to treat brain or leptomeningeal metastases of NSCLC, head and neck squamous cell carcinoma, squamous cell carcinoma, brainstem tumors, primary brain cancer, gliomas, or other cancers. The Chinese patent 201610982608.6 describes a preparation method and application of the said quinazoline derivative (I) (molecular formula C₂₃H₂₁F₃N₄O₂). As known to those skilled in the art, resistance mutations arise after treatment with osimertinib (also known as Osimertinib, AZD9291 or Tagrisso), preventing effective tumor growth inhibition by osimertinib. Therefore, developing inhibitors that can overcome these resistance mutations is highly valuable and carries significant implications.

### SUMMARY OF THE INVENTION

The objective of the present invention is to provide the use of a quinazoline compound in overcoming osimertinib resistance.

The objective of the present invention is achieved through the following technical solutions: The present invention provides the use of a quinazoline compound in the preparation of a medicament for overcoming osimertinib resistance.

As a preferred embodiment, the said quinazoline compound includes the quinazoline derivative of formula I, at least one of its salts, prodrugs, and prodrug salts, and at least one solvate, hydrate, and polymorph of the said salts.

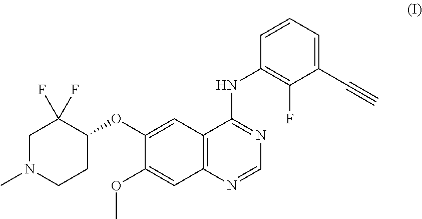 [SMILES: C#Cc1cccc(Nc2ncnc3cc(OC)c(O[C@@H]4CCN(C)CC4(F)F)cc23)c1F]

As a preferred embodiment, the said quinazoline compound includes hydrochloride, sulfate, maleate, succinate, adipate, glycolate, malate, fumarate, benzenesulfonate, benzoate, hippurate, and oxalate of the quinazoline derivative, as well as their solvates, hydrates, and polymorphs.

As a preferred embodiment, the medicament is used for the treatment of osimertinib-resistant NSCLC and NSCLC with CNS metastases.

As a preferred embodiment, NSCLC with CNS metastases includes brain metastases or leptomeningeal metastases of NSCLC.

As a preferred embodiment, the said osimertinib resistance is driven by the EGFR C797S mutation, L792H mutation, Del19/C797S dual mutations, or L858R/C797S dual mutations. Compared with the prior art, the present invention has the following advantageous effects:
1. The quinazoline derivative (I) and pharmaceutically acceptable salts thereof described in the present invention can inhibit tumor growth by overcoming osimertinib resistance especially driven by the EGFR C797S mutation, L792H mutation, Del19/C797S dual mutations, or L858R/C797S dual mutations.
2. The quinazoline derivative (I) and pharmaceutically acceptable salts thereof described in the present invention can effectively treat osimertinib-resistant NSCLC and NSCLC with CNS metastases, including brain metastasis or leptomeningeal metastasis, driven by the aforementioned resistance mutations.

### BRIEF DESCRIPTION OF DRAWINGS

The features, objectives, and advantages of the present invention will become more apparent through detailed description of the non-limiting embodiments, with reference to the accompanying drawings:

FIG. 1 shows the antiproliferative effect in Ba/F3 L858R/C797S and Del19/C797S cells compared to osimertinib.

### DETAILED DESCRIPTION

The present invention will be described in detail below with reference to specific embodiments. The following embodiments will help those skilled in the art better understand the present invention, but do not in any way limit the scope of the invention. It should be noted that, for those skilled in the art, several modifications and improvements can be made without departing from the spirit of the present invention. These are all within the scope of protection of the present invention.

### Example 1

Inhibition of Ba/F3 L858R/C797S and Del19/C797S cell growth by quinazoline derivative (I) described in the present invention compared to osimertinib in the antiproliferative activity assay

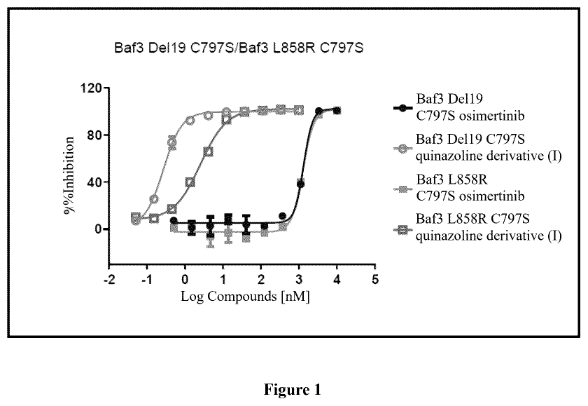

The test compounds in DMSO were added into a 384-well plate at 0.05, 0.15, 0.45, 1.37, 4.11, 12.3, 37.0, 111.1, 333.3, and 1000 nM, followed by the addition of cell suspension into the same plate at 700 cells/30 μL/well. After incubating the cells in a 5% CO₂ incubator at 37° C. for 72 hours, 25 μL of CellTiter-Glo reagent (CTG) was added to each well and the plate was gently shaken and incubated in a 5% CO₂ incubator at 37° C. (protected from light) for 30 minutes. Fluorescence was measured using the Envision reader. Antiproliferative activity was evaluated using IC₅₀ (half-maximal inhibitory concentration) and relative potency (%): Relative potency %=Osimertinib IC₅₀/Quinazoline derivative (I) IC₅₀×100%. As shown in FIG. 1, in the antiproliferative activity assay using Ba/F3 L858R/C797S and Del19/C797S cells, the quinazoline derivative (I) described in the present invention overcame osimertinib resistance and exhibited strong inhibition of cell proliferation driven by the osimertinib-resistant EGFR L858R/C797S and Del19/C797S mutations compared to osimertinib. The relative bioactivity thereof was approximately 490 times and 4900 times that of osimertinib, respectively.

| | Osimertinib | Quinazoline derivative | Relative |
|---|---|---|---|
| Cell line | IC₅₀ (nM) | (I) IC₅₀ (nM) | potency % |
| Ba/F3 L858R/C797S | 1255.0 | 2.561 | 49004.3 |
| Ba/F3 Del19/C797S | 1324.0 | 0.27 | 490370.4 |

### Example 2

Inhibition of EGFR C797S, L792H, L858R/C797S, and Del19/C797S kinases by quinazoline derivative (I) described in the present invention compared to osimertinib

The test compounds in DMSO were added into a 384-well plate at 0.017, 0.05, 0.15, 0.45, 1.37, 4.11, 12.3, 37.0, 111.1, and 333.3 nM. At room temperature, 5 μL each of kinase, peptide substrate (TK-substrate-biotin), and ATP were added to the plate and incubated at room temperature for 40 minutes. Then, 5 μL each of Sa-XL 665 HTRF detection buffer and TK-antibody-Cryptate were added to the plate and incubated at room temperature for 1 hour. Fluorescence at 615 nm (Cryptate) and 665 nm (XL665) was measured using the Envision 2104 microplate reader. Kinase inhibition was evaluated using IC₅₀ (half-maximal inhibitory concentration) and relative potency (%): Relative potency %=Osimertinib IC₅₀/Quinazoline derivative (I) IC₅₀×100%.

In the kinase inhibition assay using EGFR C797S, L792H, L858R/C797S, and Del19/C797S kinases, the quinazoline derivative (I) described in the present invention overcame osimertinib resistance and exhibited strong inhibition of the osimertinib-resistant EGFR C797S, L792H, L858R/C797S, and Del19/C797S kinases compared to osimertinib. The relative bioactivity thereof was approximately >1052 times, 49 times, 93118 times, and 498 times that of osimertinib, respectively.

| | Osimertinib | Quinazoline derivative | Relative |
|---|---|---|---|
| Kinase mutation | IC₅₀ (nM) | (I) IC₅₀ (nM) | potency % |
| EGFR C797S | >1000 | 0.95 | >105263 |
| EGFR L792H | 168.4 | 3.424 | 4918 |
| EGFR L858R/C797S | 3423 | 0.037 | 9311751.9 |
| dual mutations | | | |
| EGFR Del19/C797S | 65.2 | 0.131 | 49809.0 |
| dual mutations | | | |

The quinazoline derivative (I) described in the present invention overcomes osimertinib resistance. Compared to osimertinib, the said derivative shows strong inhibition of osimertinib-resistant EGFR C797S, L792H, L858R/C797S, and Del19/C797S mutations. It can overcome resistance caused by the EGFR C797S mutation, L792H mutation, Del19/C797S dual mutations or L858R/C797S dual mutations, thereby inhibiting tumor growth. It effectively treats osimertinib-resistant NSCLC, including NSCLC with CNS metastasis, driven by the said resistance mutations.

The above describes the specific embodiments of the present invention. It should be understood that the present invention is not limited to the above specific embodiments, and those skilled in the art can make various modifications or changes within the scope of the claims, without departing from the essence of the invention.

1. A quinazoline compound and use thereof in the preparation of a medicament for overcoming osimertinib resistance.

2. The use according to claim 1, wherein the quinazoline compound includes the quinazoline derivative of formula I, at least one of its salts, prodrugs, and prodrug salts, and at least one solvate, hydrate, and polymorph of the said salts.

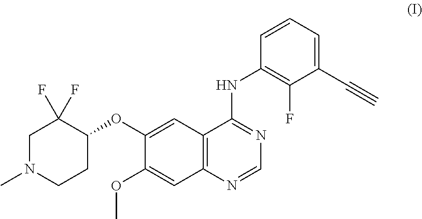 [SMILES: C#Cc1cccc(Nc2ncnc3cc(OC)c(O[C@@H]4CCN(C)CC4(F)F)cc23)c1F]

3. The use according to claim 2, wherein the salts of the quinazoline compound include hydrochloride, sulfate, maleate, succinate, adipate, glycolate, malate, fumarate, benzenesulfonate, benzoate, hippurate, or oxalate of the quinazoline derivative; the quinazoline compound further includes solvates, hydrates, or polymorphs of these salts.

4. The use according to claim 1, wherein the medicament is for the treatment of osimertinib-resistant NSCLC and NSCLC with CNS metastases.

5. The use according to claim 4, wherein NSCLC with CNS metastases includes brain metastasis or leptomeningeal metastasis of NSCLC.

6. The use according to claim 1, wherein the osimertinib resistance is driven by the EGFR C797S mutation, L792H mutation, Del19/C797S dual mutations, or L858R/C797S dual mutations.
